(19) United States
(12) Patent Application Publication
(10) Pub. No.: US 2022/0054729 A1
(43) Pub. Date: Feb. 24, 2022

(54) VASCULAR ACCESS IMPLANT AND ACCESS IMPLANT SYSTEM

(71) Applicant: B. Braun Avitum AG, Melsungen (DE)

(72) Inventors: Silvie Krause, Melsungen (DE); Kai-Uwe Ritter, Melsungen (DE); Ben Weise, Kleinwechsungen (DE); Henrik Wolff, Melsungen-Adelshausen (DE)

(21) Appl. No.: 17/413,537

(22) PCT Filed: Dec. 18, 2019
(86) PCT No.: PCT/EP2019/085855

§ 371 Date: Jun. 12, 2021

(30) Foreign Application Priority Data
Dec. 21, 2018 (DE) 10 2018 133 404.4

(51) Int. Cl. A61M 1/36; A61M 39/02

(52) U.S. Cl. CPC A61M 1/3655; A61M 2039/0258; A61M 2205/3331; A61M 39/0247

(57) ABSTRACT

A vascular access implant and system for connecting an intracorporeal blood circulation to an extracorporeal blood circulation includes an arterial fluid line for connection to a patient's artery and a venous fluid line for connection to a patient's vein. A controllable artery-vein-connection between the arterial fluid line and venous fluid line controls fluid flow between the arterial fluid line and venous fluid line. An arterial port is in fixed fluid connection with the arterial fluid line for coupling to and decoupling from a first hose of the extracorporeal blood circulation. A venous port is in fixed fluid connection with the venous fluid line for coupling to and decoupling from a second hose of the extracorporeal blood circulation.

### CROSS-REFERENCE TO RELATED APPLICATION(S)

This application is the United States national phase entry of International Application No. PCT/EP2019/085855, filed Dec. 18, 2019, and claims the benefit of priority of German Application No. 10 2018 133 404.4, filed Dec. 21, 2018. The contents of International Application No. PCT/EP2019/085855 and German Application No. 10 2018 133 404.4 are incorporated by reference herein in their entireties.

### FIELD

The present invention relates to a vascular access implant for connecting/coupling/an access/a connection of an intracorporeal blood circulation to an extracorporeal blood circulation, in particular for the (fluidic) connection to an extracorporeal blood treatment machine such as a dialysis machine, comprising an arterial fluid line/blood line adapted to be in permanent (fluidic) connection to an artery of a patient in order to draw blood from the artery, comprising a venous fluid line/blood line adapted to be in permanent (fluidic) connection to a vein of the patient to supply blood to the vein, and comprising a controllable artery-vein-connection between the arterial fluid line and the venous fluid line, which controls, in particular by means of a controllable valve, a fluid flow between the arterial fluid line and the venous fluid line.

### BACKGROUND

For connecting an intracorporeal blood circulation to an extracorporeal blood circulation, for example, an arteriovenous fistula is created as an abnormal connection between an artery and a vein of a patient. Such a connection, also known as a shunt, enables a higher blood flow through the extracorporeal blood circulation during a blood treatment.

Here, blood coming from the artery is drained into the extracorporeal blood circulation or into a first connecting tube of the extracorporeal blood circulation, then undergoes a treatment and is returned after that to the patient, in particular coming from a dialysis machine. An adequate blood flow in the extracorporeal circulation is an essential condition here for the optimal removal of the toxins contained in the blood. Depending on the extracorporeal method, very different flows are required. They range from approx. 100 ml/min for the treatment of patients in an intensive care unit to 6000 ml/min for patients in a heart-lung machine. In hemodialysis, for example, a connection of up to 400 ml/min is sufficient as a rule.

Patients with chronic kidney failure usually receive an arteriovenous fistula surgically created on their arm. This arteriovenous fistula serves as vascular access during blood treatment. A vein has very thin walls and too little blood flow. However, the veins can have a diameter suitable for frequent punctures. The arteries, on the other hand, have too little volume and are very unsuitably positioned for punctures, but have adequate blood flow. A shunt is an arterialized vein and, due to the increase in wall thickness for the frequent (vein) punctures, a suitable blood vessel with which the patient can be treated several times a week and punctured with dialysis cannulas.

Alternatively, a multi-lumen catheter can be placed in the upper or lower vena cava. If several lumina are present, they are located in a catheter that encompasses all lumina. Since there is only one vascular access, blood withdrawal and blood return must take place intermittently, i.e. alternately. This requires special instrument-based equipment of the dialysis machine. When blood is drawn in, the venous blood tube in particular must be automatically squeezed and the blood prevented from flowing back. A special control mechanism regulates the inflow and outflow phases here.

From WO 2009/033177 A1, for example, a system is known which can be used as a catheter system for dialysis. An implant is implanted under the skin of the patient in the body and connected to a blood vessel such as a vein. The implant has a single closable opening through which a catheter can be inserted and removed. An arterial flow of the blood is sucked through this opening by means of a suction pressure or negative pressure, delivered to an extracorporeal circulation, treated and then returned to the intracorporeal blood circulation by means of a flexible hose, which also protrudes through the opening into the implant and is surrounded by the sucked arterial blood. The flexible hose has a long dimension in order to extend far into the vein. Thus, the arterial blood is taken at a single blood vessel at an upstream point, treated and re-introduced downstream of the blood vessel.

WO 03/000314 A2 discloses a fistula between an artery and a vein of a patient, which can be regulated and opened (open state) or closed (closed state) by means of a controllable valve. A control unit controls the mechanical valve. Intermediate stages between the open state and the closed state are not provided.

US 2004/0133173 A1 discloses an implantable access for connecting the intracorporeal blood circulation to an extracorporeal blood circulation, here for hemodialysis, comprising a rigid structure having an arterial and a venous access as well. Both the arterial access and the venous access have an opening in a housing which can be closed mechanically.

U.S. Pat. No. 6,582,409 B1 discloses an implantable arteriovenous fistula which has a connection to an artery and a vein and is suitable for hemodialysis. This implantable arteriovenous fistula has a specially defined needle entry area within a frame that is completely received in the body. Blood can be taken from the artery and returned to the vein via special needles which are pierced through the skin of the patient and further pierced into this needle entry area. Thus, a connection to an extracorporeal blood circulation is established.

EP 1 622 657 B1 discloses a dialysis valve comprising a tube which is fluidically connected between the vein and the artery and further has a pneumatically actuatable bellows. The bellows defines an inner chamber, with the tube being arranged in the chamber, so that as the length of the bellows changes, a portion of the tube also changes its diameter correspondingly. This allows a flow between the artery and the vein to be controlled according to the adjustable diameter of the tube, and the tube functions as an adjustable throttle.

However, all these state-of-the-art systems have the disadvantage that the patient must be punctured with needles to gain access to the blood circulation, which is painful for the patient and weakens the organs and damages blood vessels, or that blood is taken from and delivered to only one blood vessel, which has a negative effect on the heart. There are also disadvantages with regard to the risk of infection. There is also a risk of dilatation of the blood vessels due to slackening of the vessel muscles, known as vasodilatation. Due to the greater resistance and an accompanying increased performance demand of the heart, the usual systems contribute to heart disease as a consequence of vasodilation. There is also always the danger that the needles loosen and lead to an unwanted loss of blood to the environment.

### SUMMARY

It is therefore the task of the invention to avoid or at least reduce the disadvantages of the state of the art and in particular to provide a vascular access implant which allows a painless and safe connection/coupling of an intracorporeal blood circulation to an extracorporeal blood circulation, which increases the safety and sterility of the connection and reduces the associated risk of infection, is self-sufficiently functional and avoids physically detrimental side effects of the dialysis treatment.

According to the invention, the vascular access implant is thus adapted to be able to be connected to the extracorporeal blood circulation for especially a blood treatment via a mechanically formed arterial (coupling) port and a mechanically formed venous (coupling) port, in a detachable, painless and safely connectable, i.e. attachable and detachable manner.

The two ports are designed to protrude in particular from the skin of the patient for detachable coupling and thus allow a simple and secure connection. Thus, the vascular access implant essentially has a first line/structure portion which is implanted in the body, separated from the environment by the skin and establishes a connection from the artery and vein to the first line/structure portion of the access implant. Furthermore, the vascular access implant has a second structure portion which projects out of the body through the skin and forms a mechanical/mechanically formed (coupling) port to which the extracorporeal blood circulation can be coupled and uncoupled. The first structure portion and the second structure portion of the vascular access implant are (mechanically and fluidically) connected. Thus, a defined interface for in particular a blood treatment is provided, which can be connected painlessly, offers a pleasant wearing comfort for the patient and guarantees a safe and sterile connection.

According to the invention, the vascular access implant thus has an arterial port which is in fixed fluid connection with the arterial fluid line and is adapted, as an arterial access, to be able to be coupled to and decoupled from a first (external) hose of the extracorporeal blood circulation. Furthermore, the vascular access implant has a venous port which is in fixed fluid connection with the venous fluid line and is adapted, as a venous access, to be able to be coupled to and decoupled from a second (external) hose of the extracorporeal blood circulation. The vascular access implant is especially designed to be fully incorporated in the body of the patient, where only the arterial port/port socket and the venous port/port socket can be connected easily, safely and quickly from the outside and preferably protrude outwards from the body through the skin of the patient.

In terms of design, the fundamental idea outlined above is essentially implemented by the fact that the vascular access implant has the arterial port and the venous port, which are each connected to the arterial or venous fluid line via a line. The other end of the line (the end of the line facing away from the arterial or venous fluid line) each has a separate or a common adapter or adapter structure for coupling, which is shaped in such a way that the adapter projects through the skin in particular and is easily accessible from outside or outside the body. By way of example, a needle can be inserted from outside into a defined needle insertion structure of the adapter in order to establish a fluid connection via the needle, or the adapter can be connected and disconnected in needle-free manner, for example by means of a Luer connection.

The terms “connectable” or “attachable and detachable” in this context mean that one end of a (first) fluid channel is directly connected to another end of a different (second) fluid channel, and these channels are fixed against each other and permanently held in this position until a desired uncoupling process is performed by a user or by a control command so that a safe and detachable fluid connection between both fluid channels is created.

Preferably, the arterial port may have an arterial connecting lock/an arterial coupling which firmly connects to the first hose with a positive fit and/or frictional fit, and/or the venous port may have a venous connecting lock which firmly connects to the second hose with a positive fit and/or frictional fit. The connecting lock ensures in each case that the line or hose is and remains securely, correctly and firmly connected to the corresponding port in fluidic manner. The connecting lock connects the hose firmly to the corresponding port, so to speak. Through the form-fitting and/or force-fitting connection, the hose can also be easily and quickly detached from the port by releasing the form-fit or force-fit.

According to an embodiment, the arterial connecting lock (the arterial coupling) and/or the venous connecting lock (the venous coupling) can connect to the respective hose in a force-fitting manner via a displaceable undercut/a displaceable undercut structure such as a hose coupling/a quick-release coupling or a Luer-Lock connection, or can connect to the corresponding hose in a form-fitting manner and/or via a magnetic or magnetizable structure, such as a magnetic coupling or a hysteresis coupling. The undercut and/or magnetizable structure offers the advantage of a detachable, painless and secure connection.

According to an aspect of the invention, the vascular access implant may comprise an autonomous energy supply/an autonomous energy supply device which provides energy for components of the vascular access implant. To operate in a self-sufficient manner, the vascular access implant needs electrical energy for its components, in particular for its electromechanical and electrotechnical components such as sensors. This energy is provided by the autonomous energy supply.

According to a further preferred, provision is made that the autonomous energy supply of the vascular access implant has an energy harvester/a nano-scale generator that converts energy via a body temperature of the patient and/or a movement of the patient and/or a pulsation of a vessel or of the controllable artery-vein-connection into electrical energy. The required electrical energy can be “gained” by the access implant by means of the energy harvester, which uses the body temperature or the movement or the pulsation as energy source. The energy harvester performs, in particular by means of the piezoelectric effect, a conversion of mechanical energy into electrical energy, and/or by means of thermoelectric generators or pyroelectric crystals, produces electrical energy from temperature differences by means of the thermoelectric effect.

In particular, the autonomous energy supply may include an energy storage device, in particular an accumulator such as a lithium ion accumulator, to store electrical energy in the autonomous energy supply of the vascular access implant. By means of the accumulator it can be ensured that the access implant provides its components with the required electrical energy over a period of time in order to act in self-sufficient manner and to fulfil its function reliably.

According to another embodiment, electrical energy can be supplied to the autonomous energy supply by means of wireless/contactless energy transmission, in particular via inductive energy transmission. It is preferred that the energy storage device of the autonomous energy supply can be recharged by contactless energy transmission. To this end, the autonomous energy supply of the access implant preferably has an antenna of a (coupling) coil in order to be charged by means of inductive coupling with a magnetic flux in the near field. Thus, the access implant can be held at a suitable transmitter with power supply (comprising a transmitter coil) at a distance of few centimeters, and the transmitter supplies the access implant with energy in a contact-less manner.

It can be useful if the state of charge of the energy storage device is indicated visually and can be read out in particular via a subcutaneous light source such as an LED, or by wireless transmission. The access implant preferably has a light source/lamp, in particular an LED or an LCD display, which is placed directly under the skin of the patient, so that the light transmittance of the skin is sufficient to display information through the skin, such as the state of charge, a coupling status or a therapy progress.

In a preferred embodiment, the arterial port and/or the venous port and/or the controllable artery-vein-connection may have a disinfection unit/sterilizing unit, which is self-disinfecting in particular, as a component of the vascular access implant, which has a biocide or anti-bacterial effect and kills bacteria and germs or at least inhibits their growth. The disinfection unit, which is integrated in the vascular access implant, serves to ensure the sterility of the arterial port and/or the venous port and/or the controllable artery-vein-connection so that the access implant can autonomously fulfil its requirements in terms of sterility. The risk of infection decreases.

Preferably, the disinfection unit/sterilizing unit is adapted to (actively) emit UV light or a plasma onto the arterial port and/or the venous port and/or the artery-vein-connection, and/or the disinfection unit may have a (passive) anti-bacterial coating and/or the disinfection unit may have a light-activatable self-disinfecting material. If the disinfection unit is designed as a UV lamp emitting UV light, it can, for example, kill bacteria and germs by means of short-wave radiation before connection to the extracorporeal blood circulation and thus actively disinfects the arterial or venous ports as well as the artery-vein connection. If the arterial and/or venous port is/are coated or equipped with an anti-bacterial coating, which acts as a disinfection unit, passive disinfection is provided for a longer period of time, especially permanently. Alternatively or additionally, the disinfection unit may have a refillable container inside the access implant, in which a disinfectant/sterilizing agent is stored which can be supplied to the arterial and/or venous port.

In a further embodiment, the vascular access implant may have an identification element and/or a transponder/transmitter, in particular an RFID chip, as a component for contact-less identification of the access implant by means of a suitable reader. The unique identification is able to ensure that the intracorporeal blood circulation is indeed connected to the correct extracorporeal blood circulation. It is also possible to send patient data in contact-less manner to the blood treatment machine in order to (automatically) set a therapy according to the blood treatment.

Furthermore, the vascular access implant may preferably have a communication unit as a component which is adapted to receive and send data in contact-less manner. The communication unit can be used to establish a data connection for instance between the extracorporeal blood treatment machine or a doctor's computer for entering updated data and the access implant. For example, after a therapy with a dialysis machine, current data can be stored in the access implant and a history can be stored in the access implant itself. During the next treatment, these values are then made available to the blood treatment machine, which evaluates the values and adapts or adjusts a treatment accordingly.

According to an aspect of the invention, the vascular access implant may comprise a port dislocation detection/port dislocation detection device as a component of the access implant at the arterial port and/or at the venous port, which is adapted to detect, in particular by means of a Hall sensor and/or a Reed sensor/reed relay, whether the arterial port or the venous port is correctly connected or whether the first or second hose is correctly connected to the arterial port or venous port, respectively. The port dislocation detection serves as a security and monitoring device of the access implant, which can detect an unintentional loosening of the hose or a blood flow without correct connection and, for example, send a corresponding control or alarm signal. This makes the connection even more secure.

In particular, the controllable artery-vein-connection, in particular by means of a proportional valve, can continuously control or discretely adjust a flow cross-section and/or a flow and/or a fluid resistance/flow resistance of the fluid connection between the arterial fluid line and the venous fluid line according to a further aspect of the invention. Preferably, the controllable artery-vein-connection additionally comprises a check valve which prevents an unwanted backflow from the venous fluid line to the arterial fluid line. The controllable artery-vein-connection or the controllable proportional valve allows an exact adjustment of the pressure and the flow in order to optimally support the blood treatment in particular. The artery-vein-connection of the access implant can be controlled discretely here, for example in order to realize two states, namely an open state in which the artery-vein-connection is completely open and the fluid connection is established, and a closed state in which the artery-vein-connection is completely closed, or it can also assume several discrete states in order to also assume intermediate states between the open state and the closed state. Alternatively, it is possible that the artery-vein-connection is continuously adjustable, for example by means of a proportional valve, in order to be able to react to hemodynamic changes. A blood flow in the artery-vein-connection can be individually adjusted and it can in particular prevent coagulation by throttling and at the same time relieve the heart. In particular, the controllable artery-vein-connection is closed if the patient is not connected to the extracorporeal blood circulation. The controllable artery-vein-connection avoids side effects during blood treatment, such as heart hypertrophy or steal syndrome.

The vascular access implant may have a flow sensor and/or a pressure sensor that measures a flow or pressure in the controllable artery-vein-connection and/or at the arterial port and/or at the venous port. The flow or pressure serves in particular to control the artery-vein-connection and is used as a basis for this. The flow sensor measures both a quantity and a sign (possible recirculation) of the blood flow and is important as a parameter for treatment. The pressure sensor measures a pressure, in particular of the artery-vein-connection, and serves, in particular together with the measured flow, as another parameter for control.

According to another preferred embodiment, the vascular access implant may have a central control unit by means of which the components of the vascular access implant are controlled. For example, the central control unit receives data from components such as sensors, processes it and sends data to components such as the communication unit. By means of the central control unit, the access implant can be controlled automatically.

It is advantageous if a controllable valve, in particular an electromechanical valve, is provided between the arterial fluid line and the arterial port and/or a check valve is provided between the venous fluid line and the venous port. In the event that the connection to the extracorporeal blood circulation is interrupted at the arterial port, the controllable valve can be quickly closed and unwanted blood loss into the environment is prevented. On the other hand, the check valve on the venous port provides a safety element that prevents blood from flowing out of the venous port. Since the venous port is only to be supplied with blood during a blood treatment but not discharged, a check valve for closure of the venous port is sufficient. In particular, the actively controlled valve of the arterial port is controlled by the central control unit.

It is advantageous if both the arterial port and the venous port are designed in a single, preferably one-piece adapter, in particular in the form of a double connector, which can be coupled only to a corresponding mating adapter. This prevents the case that a port, for example the arterial port, is connected to a hose and the other port, for example the venous port, is separated from a hose. With the single adapter, coupling to the arterial and venous port can only be made simultaneously.

According to a preferred embodiment, the vascular access implant may have a housing which, except for the arterial and venous fluid line as well as the arterial and venous port, completely sheaths/encloses/encompasses all components of the access implant, in particular the electrical components, to create a sterile barrier between the components and the body, with the housing having a biocompatible material on its outside. The housing allows to integrate various, even non-biocompatible components into the access implant, which is of immense importance in particular for electrotechnical components. Alternatively, the vascular access implant may have a (relatively) rigid biocompatible connection structure that rigidly connects the biocompatible components of the vascular access implant to each other.

In particular, the arterial and/or venous port has a sealing ring. This ensures a lossless blood connection.

In accordance with an aspect of the invention, the access implant allows to adjust a distance between the arterial fluid line and the venous fluid line. This ensures that the access implant can be used for different anatomies, since the distance between an artery and a vein is usually different for patients. The adjustable distance between an artery and a vein can be achieved, for example, by means of an artery-vein-connection which is adjustable in its length.

The invention also relates to a vascular access implant system with a vascular access implant and an extracorporeal blood circulation. Here, a vascular access implant according to the invention is inserted into the vascular access implant system, whereby an arterial mating adapter is provided at one hose end of a first hose of the extracorporeal blood circulation, which can be coupled to an arterial adapter of the arterial port in a form- and/or force-fitting manner, and/or a venous mating adapter is provided at one hose end of a second hose of the extracorporeal blood circulation, which can be coupled to a venous adapter of the venous port in a form- and/or force-fitting manner. Similar to a plug-socket connection/plug connection, the adapter and mating adapter form a coordinated system of the access implant system. The adapter fits on the corresponding mating adapter to establish a fluid connection between the adapter and the mating adapter. The adapter and mating adapter can be designed differently. For example, they can be designed as complementary quick-connect components or as a Luer-Lock connection. The system of the adapter and the mating adapter of the access implant system forms a defined interface and ensures a secure connection of the intracorporeal blood circulation to the extracorporeal blood circulation.

### BRIEF DESCRIPTION OF THE DRAWING FIGURES

The invention is explained in more detail below using a preferred embodiment with the help of accompanying Figures wherein:

FIG. 1 is a schematic top view of a vascular access implant according to the invention of an access implant system according to the invention according to a preferred embodiment;

FIG. 2 is a schematic top view of the vascular access implant of FIG. 1 placed in an arm of a patient; and

FIG. 3 is a schematic diagram view of the vascular access implant and the access implant system from FIG. 1.

### DETAILED DESCRIPTION

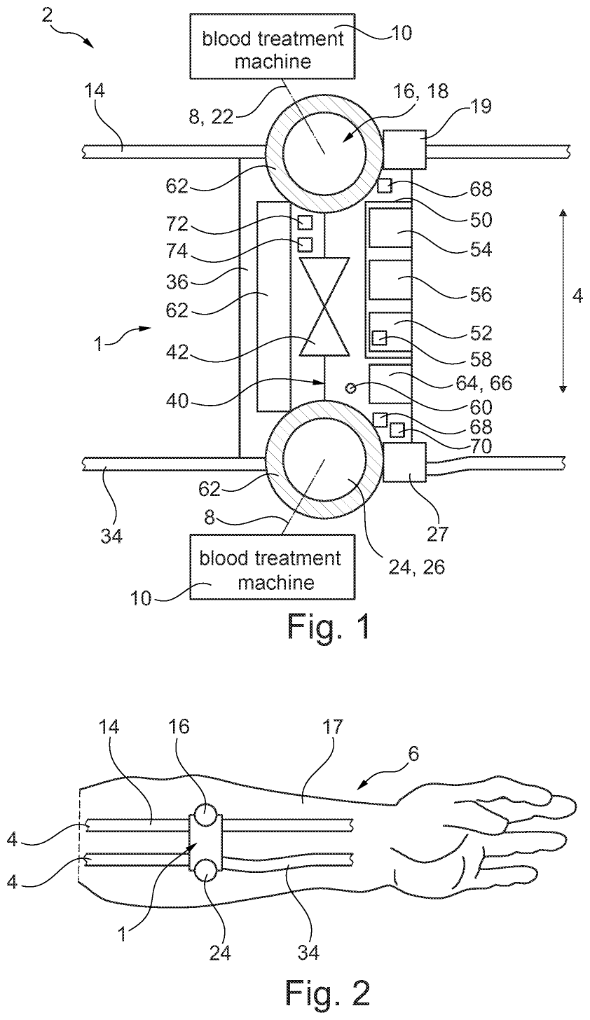

FIGS. 1 to 3 show a vascular access implant 1 according to the invention and an access implant system 2 of the invention according to a preferred embodiment comprising a variety of components.

The access implant 1 serves as an interface between an intracorporeal blood circulation 4 of a patient 6 and an extracorporeal blood circulation 8 in order to draw blood from the patient 6, treat it with a blood treatment machine 10 in the form of a dialysis machine and then feed it back to the patient 6. With the extracorporeal blood circulation 8, various blood treatments can be performed, such as hemodialysis, hemodiafiltration, ultrafiltration or (therapeutic) apheresis to remove toxins from the blood.

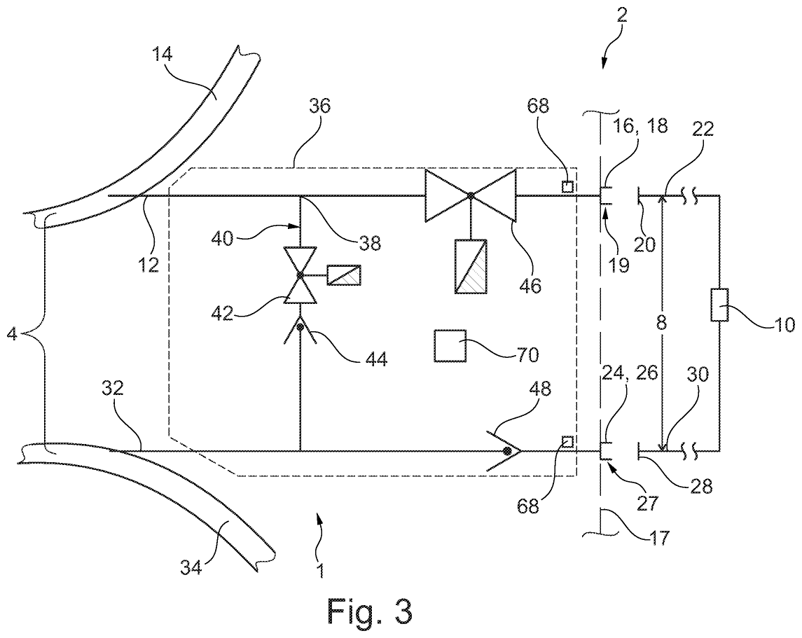

The access implant 1 has an arterial fluid line 12 (see FIG. 3), for example in the form of a flexible arterial silicone line or a line made of a suitable vascular replacement material, which protrudes into an artery 14 of the patient 6 and is fluidly connected to it. Alternatively, the arterial fluid line 12 can also be firmly sutured to the artery 14. The arterial fluid line 12 can be used to draw off or withdraw/remove a quantity of blood from the artery 14 of the patient 6.

The arterial fluid line 12 conveys the extracted blood to a rigid arterial port 16. This arterial port 16 projects through a skin 17 of the patient 6, penetrates the skin barrier, so to speak, so that the arterial port 16 is easily accessible from outside (outside the body) and can be coupled to and decoupled from the extracorporeal blood circulation 8. The arterial port 16 features an arterial adapter 18 or is designed as an arterial adapter 18, which is adapted to be able to be coupled to and decoupled from an arterial mating adapter 20 of the extracorporeal blood circulation 8 in a form-fitting and/or magnetic manner (i.e. in a force-fitting manner) (see FIG. 3). The arterial adapter 18 is connected to the arterial fluid line 12 in fluid-tight manner and forms, so to speak, the terminal (blood) outlet of the access implant 1 to the extracorporeal blood circulation 8.

The arterial adapter 18 of the intracorporeal blood circulation 4 and the arterial mating adapter 20 of the extracorporeal blood circulation 8 together form a coordinated system of the access implant system 2 and a defined interface. The arterial adapter 18 fits on the corresponding arterial mating adapter 20 to create a lossless, simple and detachable fluid connection between the adapter 18 and the mating adapter 20. The mating adapter 20 forms a terminal portion of a first (arterial) hose 22 of the extracorporeal blood circulation 8. The adapter 18 as one end of the (first) fluid channel (of the intracorporeal blood circulation 4) is thus directly connected to the mating adapter 20 as another end of the (second) fluid channel (of the extracorporeal blood circulation 8) and fixed against each other and permanently held in this position until a desired decoupling process is performed by a user. This creates a safe and detachable fluid connection between both blood circulations 4, 8. The arterial adapter 18 functions as an arterial connecting lock 19. A magnetic maximum force of attraction is usually sufficient to realize an arterial connecting lock 19 in a force-fitting manner. In particular, the arterial connecting lock 19 is designed in such a way that in the event of a strong pull on the mating adapter 20 and thus on the adapter 18, no matter in which direction, the connection is released, thus minimizing the risk of the access implant 1 being torn out with greatest damage consequence.

Analogous to the arterial side of the access implant 1, the vascular access implant 1 also has a rigid venous port 24 on its venous side with analogous features of a venous adapter 26, which can be connected to a corresponding venous mating adapter 28 of a second hose 30 of the extracorporeal blood circulation 8 in a force- and/or form-fitting manner. The venous adapter 26 thus forms a venous connecting lock 27. The cleaned or treated blood can be returned to the access implant 1 via the connection with the venous port 24. The blood flows to a venous fluid line 32 connected to the venous port 10 and realized in the form of a flexible venous silicone line that extends into a vein 34 of the patient 6 and is fluidly connected to it. Thus, a fluid connection is established between the second hose 30 and the vein 34 of the patient 6. The arterial port 16 and the venous port 24 create a fluid circuit that connects the intracorporeal blood circulation 4 of the patient 6 with the extracorporeal blood circulation 4.

The way of function of the vascular access implant 1 according to the preferred embodiment is explained below. So instead of piercing a cannula through the skin 17 of the patient 6 into the vessel (artery/vein) of the patient 6 during each treatment of the patient 6 and thus creating a blood collection site to drain the blood for an extracorporeal blood treatment, and also performing a second puncture to return the purified blood to the vein of the patient 6, as is the case with a commonly used shunt, the access implant 1 provides a fully implantable interface device which is permanently connected to both the artery 14 and the vein of the patient 34 and which provides a defined common port with two separate fluid connections or two separate ports (arterial port 16 and venous port 24) to the extracorporeal blood circulation 8. The extracorporeal blood circulation 8 can be (detachably) coupled to the access implant 1 in painless manner, without having restrictions such as the number of attachments and detachments or a change of location, as is the case with cannula punctures (vascular damage).

The access implant 1 also has a housing 36 which has its outside designed with biocompatible materials and can be permanently and completely implanted in the body of the patient 6 due to its structural design. Only the arterial port 16 or the arterial adapter 18 and the venous port 24 or the venous adapter 26 protrude from the skin 15 of the patient. The housing 36 encloses, except for the projecting arterial fluid line 12, the projecting arterial port 16, the projecting venous fluid line 32 and the projecting venous port 24, the access implant 1.

In addition, the vascular access implant 1 has a branch 38 starting from the arterial fluid line 12, which is connected to the venous fluid line 32 via a controllable artery-vein-connection 40 in order to short-circuit the artery 14 with the vein 34, similar to a bypass. In the artery-vein-connection 40, a controllable electromechanical proportional valve 42 is interposed to continuously control the flow in the artery-vein-connection 40. The proportional valve 42 can be continuously closed from an open position and then reopened again, which also realizes states between an open position and a closed position. Via the proportional valve 42, a resistance or flow in the artery-vein-connection 40 can be changed within milliseconds in order to react to hemodynamic changes. A blood flow in the artery-vein-connection 40 can thus be individually adapted, and it is possible to prevent a coagulation by having a throttling and at the same time relieve the heart. If the patient 6 is connected to the blood treatment machine 10 using the extracorporeal blood circulation 8, the controllable artery-vein-connection 40 is opened, and if the patient 6 is no longer connected to the extracorporeal blood circulation 8, it is closed.

In order to prevent a recirculation of blood into the artery 14 in the artery-vein-connection 40, a check valve 44 is also provided in the artery-vein-connection 40, which only permits a flow direction from the artery 14 to the vein 34.

In front of the arterial port 8 or upstream of it, a controllable electromechanical valve 46 is provided in the arterial fluid line 12, which switches over between an open state and a closed state. Alternatively, the controllable valve 46 can also change a flow in continuous manner. The controllable valve 46 is designed to immediately interrupt the blood flow when the arterial mating adapter 20 is decoupled from the arterial port 16 to prevent uncontrolled discharge into the environment.

A check valve 48 in the venous fluid line 32 is also provided in front of the venous port 24. This check valve does not necessarily have to be actively operable, as it closes the venous port 24 from the environment depending on the pressure in the event of uncoupling. The venous port 24 only allows blood to flow into the access implant 1 by means of the check valve 48. The check valve 48 thus securely closes the venous port 24 and ensures that there is no unwanted exit point of the blood system of the patient 6.

In order to be able to act autonomously and independently or to be capable of being actuated, the access implant 1 comprises an autonomous energy supply 50 which provides electrical energy for the (electrotechnical) components of the access implant 1. On the one hand, the autonomous energy supply 50 comprises an energy storage device 52 in the form of a rechargeable lithium ion battery to store electrical energy over a longer period of time and, on the other hand, the autonomous energy supply 50 has a first energy harvester 54 in the form of a thermoelectric generator which converts temperature differences into electrical energy by means of the thermoelectric effect and feeds it to the energy storage device 52, and a second energy harvester 56 which can convert movement energy of the patient 6 into electrical energy and feeds it to the energy storage device 52. With the two energy harvesters 54, 56, the autonomous energy supply 50 of the access implant 1 can be continuously supplied with electrical energy without the need to connect it to an external charging station. In order to be able to charge the energy storage device 52 of the autonomous energy supply 50 independently of the energy harvesters 54, 56, if necessary, the autonomous energy supply 50 has an antenna of a coupling coil or charging coil 58 so that it can also be charged wirelessly in the near field if required by means of inductive energy transmission. This allows the access implant 1 to be held to a complementary external coupling coil at a distance of a few centimeters in order to charge the energy storage device 52 via the charging coil 58 in inductive and contact-less manner.

The access implant 1 also has an indicator 60 in the form of an LED to indicate the state of charge of the energy storage device 52. The LED has sufficient luminosity to be visually perceived through the skin 17 of the patient 6. For example, the state of charge of the energy storage device 52 can be displayed in the form of the red flashing LED when the energy level is low.

In order to sterilize the arterial port 16 and the venous port 24 for coupling/connecting, the access implant 1 has an anti-bacterial disinfection unit 62 in the form of a UV lamp (emitter) as a component of the access implant 1. The disinfection unit 62 can disinfect or sterilize surfaces of both ports 16 and 24 by emitting UV light. In addition, the arterial and venous ports 16, 24 have a material with anti-bacterial effect or an anti-bacterial coating to achieve a long-lasting passive disinfection of the ports 16, 24. The artery-vein-connection 40 can also be irradiated and disinfected with UV light from the disinfection unit 62.

Data can be transferred to the extracorporeal blood treatment machine 10 by means of a communication unit 64 as a further component, in particular by means of a Bluetooth® or WLAN connection, in order to optimally adjust a therapy. Data can also be transferred to the access implant via the communication unit 64. The communication unit 64 is thus designed for wireless communication with other machines or systems.

An identification element 66 (as a further component) in the form of an RFID chip or an NFC coil is also connected to the communication unit 64 within the housing 36 of the vascular access implant 1. Using the identification element 66, the patient 6 can be uniquely identified by other machines. For example, in addition to the data transmitted via the communication unit 64, an external reader of the blood treatment machine 10 can wirelessly read out the data of the identification element 66 and adapt and adjust a therapy to the patient 3 accordingly. Since, for example, an NFC coil only has a very short range, data security can be increased since only data in the near field is transmitted. Reading the patient data by an unauthorized person is made more difficult, as the data would have to be tapped in the immediate vicinity. In particular, the data in the access implant 1 can also be encrypted, whereby preferably the communication unit 64 or the central control unit 70 performs the encryption. The blood treatment machine 10, which knows the encryption key, can then correspondingly decrypt the data transmitted in encrypted form. Similarly, the access implant 1 can also decrypt encrypted data from the blood treatment machine 10.

In order to determine a correct connection status at the arterial and venous port 16, 24, the access implant 1 has a port dislocation detection 68 (as a further component), which can detect by means of the Reed sensor and/or Hall sensor whether the respective mating adapter 20, 28 is correctly connected to the adapter 18, 26. The port dislocation detection 68 measures a magnetic or a changing magnetic flux and determines whether there is a correct coupling to the respective port 16, 24. A correct connection is of enormous importance, as it functions as a safety device and detects the intentional or unintentional loosening of the hose 22, 30 and then interrupts a fluid connection by means of the controllable valves 46 in order to avoid blood loss. In particular, the check valve 48 can also be (electromechanically) closable. If, for example, only the arterial hose 22 comes loose unintentionally, the check valve 48 can be closed, triggering a pressure alarm on the blood treatment machine 10 and stopping a blood pump. Alternatively, the access implant 1 can also close both valves 46, 48 and send a control command or alarm command via the communication unit 64 to the blood treatment machine 10 which then stops the blood treatment and displays an alarm signal or error information associated with the error on the display.

The vascular access implant 1 also has a central control unit 70, which processes received data/signals and can drive all (electrotechnical) components of the access implant 1. The control unit 70 is thus the central node of the access implant 1 for all electrotechnical components. The central control unit 70 controls the proportional valve 42 and receives data from it regarding a current (valve) state, controls the controllable valve 46 and also receives data from the latter on its state, controls the autonomous energy supply 50 with the energy storage device 52, the two energy harvesters 54, 56 and the charging coil 58 and receives data from it on, for example, a current state of charge, a current maximum charge capacity, an energy production and an energy consumption by the components, controls the indicator 60 and optionally an acoustic signal generator, controls the disinfection unit 62 and, if any, receives error messages, for example if the UV lamp is defective, controls the communication unit 64 and receives data from it, controls the identification element 66, if necessary, and receives data from the port dislocation detection 68. In addition, the control unit 70 stores relevant data such as patient data, therapy data, component data and reference data in its memory.

The artery-vein-connection 40 has a pressure sensor 72 and a flow sensor 74, which measures the pressure and the flow in the artery-vein-connection 40 and forwards them to the central control unit 70 as data. The data and parameters of the pressure sensor 72 and the flow sensor 74 form the basis for controlling the proportional valve 42 of the controllable artery-vein-connection 40, which can be used to avoid side effects of a blood treatment such as cardiac hypertrophy.

With the access implant 1 according to the invention or the access implant system according to the invention, the patient is provided with a device or system for pain-free coupling with increased safety, reduced risk of infection, improved patency and better therapy conditions with less vasodilation, lower risk of heart disease and lower susceptibility to unwanted blood loss.

1. A vascular access implant for connecting an intracorporeal blood circulation to an extracorporeal blood circulation, the vascular access implant comprising:
an arterial fluid line adapted to be permanently connected to an artery of a patient;
a venous fluid line adapted to be permanently connected to a vein of the patient;
a controllable artery-vein-connection between the arterial fluid line and the venous fluid line that controls a fluid flow between the arterial fluid line and the venous fluid line;
an arterial port which is in fixed fluid connection with the arterial fluid line and is adapted, as an arterial access, for coupling to and decoupling from a first hose of the extracorporeal blood circulation; and
a venous port which is in fixed fluid connection with the venous fluid line and is adapted, as a venous access, for coupling to and decoupling from a second hose of the extracorporeal blood circulation.

2. The vascular access implant according to claim 1, wherein:
the arterial port comprises an arterial connecting lock that firmly connects to the first hose by a positive fit and/or frictional fit, and/or
the venous port comprises a venous connecting lock that firmly connects to the second hose by a positive fit and/or frictional fit.

3. The vascular access implant according to claim 2, wherein the arterial connecting lock and/or the venous connecting lock connect to the first hose and/or second hose, respectively, via an undercut structure in a form-fitting manner or via a magnetic or magnetizable structure in a force-fitting manner.

4. The vascular access implant according to claim 1, wherein the vascular access implant comprises an autonomous energy supply that provides energy for components of the vascular access implant.

5. The vascular access implant according to claim 4, wherein the autonomous energy supply comprises an energy harvester that converts energy of a body temperature of the patient and/or of a movement of the patient and/or of a pulsation of a vessel or the controllable artery-vein-connection into electrical energy and supplies it to the autonomous energy supply.

6. The vascular access implant according to claim 4, wherein the autonomous energy supply comprises an energy storage device for storing electrical energy in the autonomous energy supply of the vascular access implant.

7. The vascular access implant according to claim 4, wherein electrical energy can be supplied to the autonomous energy supply by wireless/contact-less energy transmission.

8. The vascular access implant according to claim 7, wherein the autonomous energy supply comprises an antenna of a coupling coil for wireless energy transmission, by which energy is supplied to the autonomous energy supply in a contact-less manner via inductive coupling with a magnetic flow in a near field.

9. The vascular access implant according to claim 1, wherein the arterial port and/or the venous port and/or the controllable artery-vein-connection comprise a disinfection unit as a component of the vascular access implant hich that has an anti-bacterial effect.

10. The vascular access implant according to claim 9, wherein:
the disinfection unit emits UV light or a plasma to at least one of the arterial port, the venous port, and the controllable artery-vein-connection; and/or
the disinfection unit comprises an anti-bacterial coating or an anti-bacterial material and/or comprises a self-disinfecting material adapted to be activated by light.

11. The vascular access implant according to claim 1, further comprising:
an identification element for contact-less identification by means of a reader; and/or
a communication unit adapted to receive and send data in a contact-less manner.

12. The vascular access implant according to claim 1, wherein the vascular access implant comprises a port dislocation detection at the arterial port and/or at the venous port that is adapted to detect whether the arterial port and the venous port are correctly coupled.

13. The vascular access implant according to claim 4, wherein the autonomous energy supply supplies energy to at least one of the disinfection unit, the identification element, the communication unit, and the port dislocation detection.

14. A vascular access implant system comprising:
a vascular access implant according to claim 1, and
an extracorporeal blood circulation,
the vascular access implant system further comprising at least one of:
an arterial mating adapter provided at one hose end of the first hose of the extracorporeal blood circulation, said arterial mating adapter configured for coupling to the arterial port in a form- and/or force-fitting manner; and
a venous mating adapter provided at one hose end of the second hose of the extracorporeal blood circulation, said venous mating adapter configured for coupling to the venous port in a form- and/or force-fitting manner.
